SEQUENCE LISTING (8 sequences)

SEQ ID NO: 1 - DNA, 20 bp, synthetic construct
  misc_feature 1..20: note = Synthetic: Forward primer
  source 1..20: mol_type = other DNA
gcccatcctctgtgactcat

SEQ ID NO: 2 - DNA, 20 bp, synthetic construct
  misc_feature 1..20: note = Synthetic: Reverse primer
  source 1..20: mol_type = other DNA
aggccacaggtattttgtcg

SEQ ID NO: 3 - DNA, 21 bp, synthetic construct
  misc_feature 1..21: note = Synthetic: Forward primer
  source 1..21: mol_type = other DNA
ccagttgccttcttgggactg

SEQ ID NO: 4 - DNA, 23 bp, synthetic construct
  misc_feature 1..23: note = Synthetic: Reverse primer
  source 1..23: mol_type = other DNA
caggtctgttgggagtggtatcc

SEQ ID NO: 5 - DNA, 24 bp, synthetic construct
  misc_feature 1..24: note = Synthetic: Forward primer
  source 1..24: mol_type = other DNA
aaaattcgagtgacaagcctgtag

SEQ ID NO: 6 - DNA, 24 bp, synthetic construct
  misc_feature 1..24: note = Synthetic: Reverse primer
  source 1..24: mol_type = other DNA
cccttgaagagaacctgggagtag

SEQ ID NO: 7 - DNA, 20 bp, synthetic construct
  misc_feature 1..20: note = Synthetic: Forward primer
  source 1..20: mol_type = other DNA
tgtgtccgtcgtggatctga

SEQ ID NO: 8 - DNA, 25 bp, synthetic construct
  misc_feature 1..25: note = Synthetic: Reverse primer
  source 1..25: mol_type = other DNA
caccaccttcttgatgtcatcatac

(19) United States
(12) Patent Application Publication
(10) Pub. No.: US 2025/0360155 A1
(43) Pub. Date: Nov. 27, 2025

(54) METHOD OF TREATING SPINAL CORD INJURY AND COMPOSITION FOR USE THEREIN

(71) Applicant: ACADEMIA SINICA, Taipei City (TW)

(72) Inventors: Yijuang CHERN, Taipei City (TW); Jim-Min FANG, Taipei City (TW); Chun-Jung LIN, Taipei City (TW); Chiao-Shin LU, Taipei City (TW); Kuan-Yu CHEN, New Taipei City (TW)

(73) Assignee: ACADEMIA SINICA, Taipei City (TW)

(21) Appl. No.: 18/836,465

(22) PCT Filed: Feb. 9, 2023
(86) PCT No.: PCT/US2023/062295

§ 371 Date: Aug. 7, 2024

Related U.S. Application Data
(60) Provisional application No. 63/308,720, filed on Feb. 10, 2022.

(51) Int. Cl. A61K 31/7076

(52) U.S. Cl. CPC A61K 31/7076

(57) ABSTRACT

A method of treating spinal cord injury is provided, including administrating to a subject a compound of formula (I), (II) or (III): or a pharmaceutically acceptable salt thereof, wherein X is halogen. A composition for use in a method of treating spinal cord injury, including administering to a subject in need thereof the composition comprising a compound of formula (I), (II) or (III) as shown above is also provided.

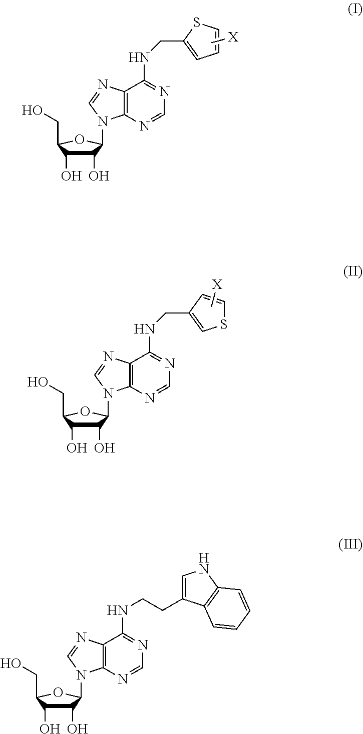

### CROSS REFERENCE TO RELATED APPLICATIONS

This application is the National Phase of PCT/US2023/062295 filed on Feb. 9, 2023, which claims priority under 35 U.S.C. § 119(e) to U.S. Provisional Application No. 63/308,720 filed on Feb. 10, 2022, all of which are hereby expressly incorporated by reference into the present application.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 16, 2023, is named “5025-0402PW01.xml” and is 10,989 bytes in size. The sequence listing contained in this .XML file is part of the specification and is hereby incorporated by reference herein in its entirety.

### FIELD OF INVENTION

The present disclosure relates to a method of treating spinal cord injury and a composition for use in a method of treating spinal cord injury.

### BACKGROUND OF THE INVENTION

There are approximately 27 million individuals living with the spinal cord injury (SCI) worldwide, and close to 1 million new cases per year. Thus, SCI is a devastating neurologic disorder that constitutes a considerable portion of the global injury burden and the number of individuals living with SCI is expected to increase in view of population growth.¹ SCI mostly occurs from a sudden and traumatic impact on the spine that dislocates or fractures the vertebrae. Following the initial mechanical impact that changes the vascular tissue and blood supply to the area of SCI, the secondary injury may include hemorrhage, ischemia, infiltration of immune cells, and the release of inflammatory cytokines, and ultimately neuronal death.² SCI is generally considered as an irreversible neurological impairment and its treatment is still an unmet medical need.

Neuroinflammation is a key component for the secondary injury mechanisms in SCI.³ Interleukin-1β (IL-1β), tumor necrosis factor alpha (TNF-α), and interleukin-6 (IL-6) are considered to be the critical mediators of the post-traumatic inflammatory reaction that may lead to neuronal death.⁴ Also, the activation of microglia and astrocytes are notable responses of neuroinflammation. While astrogliosis has been regarded as a detrimental effect for SCI, emerging studies have suggested that reactive astrocytes also play a protective role in SCI.⁵ Reactive astrocytes can be developed into two types, A1 and A2, respectively. A1 astrocytes have been reported to exert cytotoxic effects on local neurons and oligodendrocytes, whereas A2 astrocytes can promote neuron outgrowth and survival, and contribute to synapse formation and tissue repair.

### SUMMARY OF THE INVENTION

The present invention provides a new method of treating spinal cord injury (SCI) by using the compounds of adenosine analogues, an inhibitor of equilibrative nucleoside transporter 1 (ENT1), on the pathogenesis and functional recovery of SCI.

In an aspect of the present invention, a method of treating spinal cord injury, including administering to a subject in need thereof a compound of formula (I), (II) or (III):

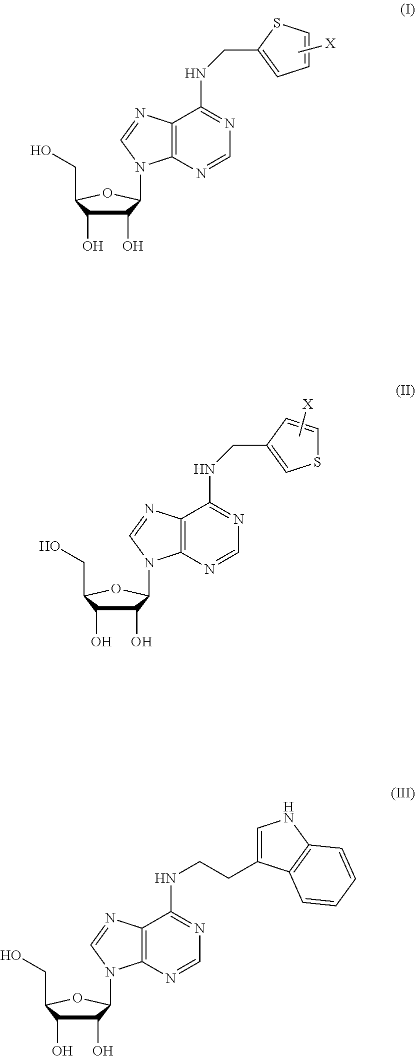

a pharmaceutically acceptable salt thereof, or a composition thereof, wherein X is halogen.

In another aspect of the present invention, a composition for use in a method of treating spinal cord injury, including administering to a subject in need thereof the composition including a compound of formula (I), (II) or (III) as shown above.

Preferably, the compound is selected from the group consisting of N⁶-[(3-halothien-2-yl)methyl]adenosine, N⁶-[(4-halothien-2-yl)methyl]adenosine, and N⁶-[(5-halothien-2-yl)methyl]adenosine. More preferably, the compound is selected from the group consisting of N-[(5-iodothien-2-yl)methyl]adenosine, M-[(4-iodothien-2-yl)methyl]adenosine, M-[(3-iodothien-2-yl)methyl]adenosine, N⁶-[(5-bromothien-2-yl)methyl]adenosine, M-[(4-bromothien-2-yl)methyl]adenosine, M-[(3-bromothien-2-yl)methyl]adenosine, M-[(5-chlorothien-2-yl)methyl]adenosine, N⁶-[(4-chlorothien-2-yl)methyl]adenosine, and N⁶-[(3-chlorothien-2-yl)methyl]adenosine.

Preferably, the compound is selected from the group consisting of N-[(2-halothien-3-yl)methyl]adenosine, N⁶-[(4-halothien-3-yl)methyl]adenosine, and N⁶-[(5-halothien-3-yl)methyl]adenosine. More preferably, the compound is selected from the group consisting of N-[(2-iodothien-3-yl)methyl]adenosine, M-[(4-iodothien-3-yl)methyl]adenosine, M-[(5-iodothien-3-yl)methyl]adenosine, N⁶-[(2-bromothien-3-yl)methyl]adenosine, M-[(4-bromothien-3-yl)methyl]adenosine, M-[(5-bromothien-3-yl)methyl]adenosine, M-[(2-chlorothien-3-yl)methyl]adenosine, N⁶-[(4-chlorothien-3-yl)methyl]adenosine, and N⁶-[(5-chlorothien-3-yl)methyl]adenosine.

Preferably, the compound, a pharmaceutically acceptable salt thereof, or a composition thereof is administered by an oral, intravenous, intramuscular, subcutaneous, intraperitoneal, or topical route.

Preferably, the composition further includes a pharmaceutically acceptable carrier, excipient or vehicle.

Therefore, the present invention at least provides the following advantages:
1. The claimed method can significantly reduce the mRNA levels of IL-1β, IL-6 and TNF-α and the activation of astrocytes and microglia/macrophage at the perilesional site of the spinal cord.
2. The claimed method can ameliorate neuroinflammation and neuronal damage of SCI, which improves functional recovery.

### BRIEF DESCRIPTION OF THE DRAWINGS

FIG. 1 is a schematic diagram which illustrates J4 was intraperitoneally injected three days before injury and then continuously injected daily for 14 days following the spinal cord injury according to an embodiment of the present invention.

FIG. 2 illustrates Basso mouse scale (BMS) of J4-treated mice at different days following the spinal cord contusion according to an embodiment of the present invention.

FIG. 3 illustrates representative images of footprint analysis of J4-treated mice at 14 days post-injury (14 DPI) and the quantitative results thereof according to an embodiment of the present invention.

FIG. 4 illustrates Nissl-stained of sagittal section of spinal cord of J4-treated mice on 14 DPI and the quantitative results of the lesion volume thereof according to an embodiment of the present invention. Scale bar is 500 μm.

FIG. 5 illustrates representative images of NeuN (green) in the perilesional area of J4-treated mice and the quantitative results thereof according to an embodiment of the present invention. Scale bar is 50 μm.

FIG. 6 illustrates the protein level of NeuN on 14 DPI at T9-T11 of J4-treated mice and the quantitative densitometric analysis of these proteins according to an embodiment of the present invention.

FIG. 7 illustrates Protein level of c-caspas3 on 14 DPI at T9-T11 of J4-treated mice and the quantitative densitometric analysis of these proteins according to an embodiment of the present invention.

FIG. 8 illustrates the mRNA levels of IL-10 (A), IL-6 (B), and TNF-α (C) at perilesional area of J4-treated mice at 14 days post-injury according to an embodiment of the present invention.

FIG. 9 illustrates representative images of Gfap (green) and Iba-1 (E; red) at the perilesional area of J4-treated mice and the quantitative results thereof according to an embodiment of the present invention. Scale bar is 100 μm.

FIG. 10 illustrates representative images of Iba-1 (red) at the perilesional area of J4-treated mice and the quantitative results thereof according to an embodiment of the present invention. Scale bar is 100 μm.

FIG. 11 illustrates immunoblots of C3 in J4-treated mice and the quantifications of densitometry of the proteins thereof according to an embodiment of the present invention.

FIG. 12 illustrates immunoblots of S100a10 in J4-treated mice and the quantifications of densitometry of the proteins thereof according to an embodiment of the present invention.

FIG. 13 illustrates immunoblots of TGF-β in J4-treated mice and the quantifications of densitometry of the proteins thereof according to an embodiment of the present invention.

FIG. 14 illustrates that JMF1907 treatment can significantly improve the motor function in terms of BMS score (A) and the stride length (B) of mice with SCI according to an embodiment of the present invention.

FIG. 15 illustrates that JMF1907 treatment significantly reduced the lesion of SCI as examined by Nissl staining and Pdgf§ expression according to an embodiment of the present invention.

FIG. 16 illustrates that JMF1907 treatment can ameliorate neuroinflammation in SCI by reducing the expression of Gfap according to an embodiment of the present invention.

FIG. 17 illustrates that JMF1907 treatment can ameliorate neuroinflammation in SCI by reducing the expression of Iba-1 and CD38 according to an embodiment of the present invention.

### DETAILED DESCRIPTION

In one embodiment, a method of treating spinal cord injury (SCI) is provided, including administrating to a subject a compound of formula (I), (II) or (III):

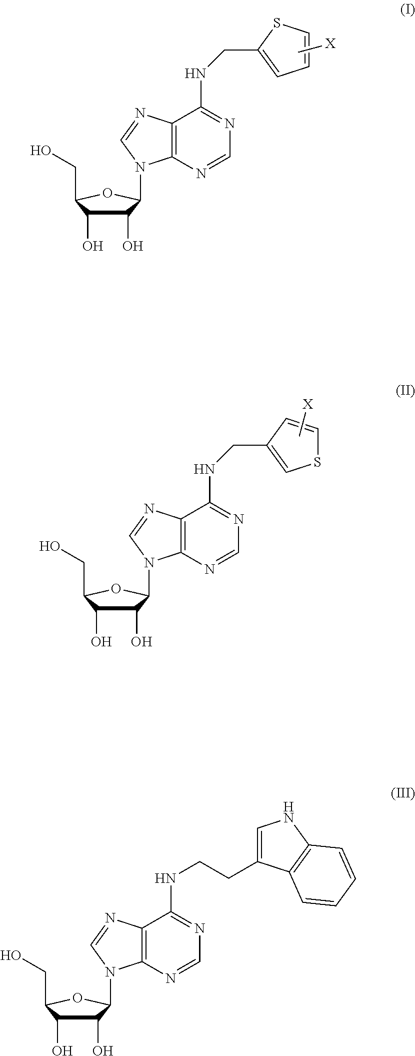

a pharmaceutically acceptable salt thereof, or a composition thereof, wherein X is halogen. Wherein, the compound of formula (III) is also callas “JMF 1907” herein.

In another embodiment, the compound may be selected from N⁶-[(3-halothien-2-yl)methyl]adenosine, N⁶-[(4-halothien-2-yl)methyl]adenosine, and N⁶-[(5-halothien-2-yl)methyl]adenosine. Preferably, the compound is N⁶-[(5-iodothien-2-yl)methyl]adenosine, N⁶-[(4-iodothien-2-yl)methyl]adenosine, N⁶-[(3-iodothien-2-yl)methyl]adenosine, N⁶-[(5-bromothien-2-yl)methyl]adenosine (also called “JMF3464” or “J4”), N⁶-[(4-bromothien-2-yl)methyl]adenosine, N⁶-[(3-bromothien-2-yl)methyl]adenosine, N⁶-[(5-chlorothien-2-yl)methyl]adenosine (also called “JMF3818”), N⁶-[(4-chlorothien-2-yl)methyl]adenosine, N⁶-[(3-chlorothien-2-yl)methyl]adenosine, or a combination thereof.

In another embodiment, the compound may be selected from N⁶-[(2-halothien-3-yl)methyl]adenosine, N⁶-[(4-halothien-3-yl)methyl]adenosine, and N⁶-[(5-halothien-3-yl)methyl]adenosine. Preferably, the compound is N⁶-[(2-iodothien-3-yl)methyl]adenosine, N⁶-[(4-iodothien-3-yl)methyl]adenosine, N⁶-[(5-iodothien-3-yl)methyl]adenosine, N⁶-[(2-bromothien-3-yl)methyl]adenosine, N⁶-[(4-bromothien-3-yl)methyl]adenosine, N⁶-[(5-bromothien-3-yl)methyl]adenosine N⁶-[(2-chlorothien-3-yl)methyl]adenosine, N⁶-[(4-chlorothien-3-yl)methyl]adenosine, or N⁶-[(5-chlorothien-3-yl)methyl]adenosine, or a combination thereof.

In one embodiment, the compound, a pharmaceutically acceptable salt thereof, or a composition thereof is administered by an oral, intravenous, intramuscular, subcutaneous, intraperitoneal, or topical route.

### EXAMPLES

### Materials and Methods

### Animals

### Induction of Spinal Cord Contusion

Mice were anesthetized by intramuscular injection of Zoletil® (50 mg/kg; tiletamine hydrochloride and zolazepam hydrochloride) and Rompun® (12 mg/kg; xylazine hydrochloride) before the surgery. After laminectomy on the T9-T10 vertebral column, the spinal cord was exposed and contused by a 10 g rod with an impact head of 1.2 mm diameter at the height of 6.25 mm above the T10 spinal cord to allow it to drop using an Impactor model-II spinal cord contusion system (RWD6809911) to cause the contusion injury. In the sham group, surgery procedures were identical except for the spinal contusion. After the injury, the wound was quickly sutured using 4.0 silk thread (Unik Taiwan; New Taipei city, Taiwan). The bladders of mice were manually and gently massaged once a day to avoid retention of urine until the reflexive control of micturition was restored. Cefazolin (300 mg/kg) was given for consecutive three days after surgery to prevent infection. Fourteen days after the surgery, mice were sacrificed for analyses.

### Pharmacological Treatment

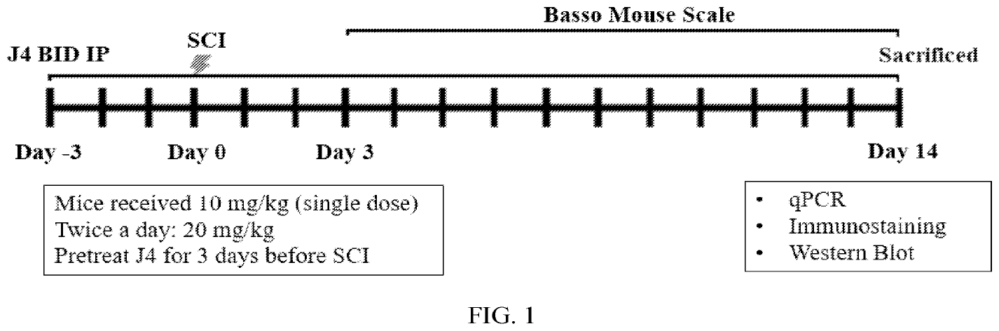

In one example for the treatment of J4, which can be used as an ENT1 inhibitor, male C57BL/6J mice (8-12 weeks old) were divided into three groups: the sham group, the SCI+J4 group and the SCI+vehicle group. Please refer to FIG. 1. In the J4 group, mice were pretreated with 10 mg/kg J4 twice a day for three days before the injury. Following the SCI, J4 (10 mg/kg) was administered to mice twice a day for consecutive 14 days. In vehicle group, mice were treated with 5% DMSO, instead of J4, following the same protocol of the J4-treated group. The sham group received the same treatment as the vehicle group. All mice were maintained on a 12 h light/dark schedule and given food and water ad libitum in the Laboratory Animal Center (an AAALAC accredited experimental animal facility) at National Taiwan University. Procedures involving animals were conducted according to the ARRIVE (Animal Research: Reporting In Vivo Experiments) guidelines.

In another example for the treatment of JMF 1907 (i.e. the compound of formula (III)), male C57BL/6J mice (8-12 weeks old) were divided into three groups: the JMF1907 group, the methylprednisolone (MPSS) group and the vehicle group. Wherein, JMF1907 (10 mg/kg twice daily for 14 days), methylprednisolone (30 mg/kg 10 min after crush, then at 2, 4, and 6 h after crush), or vehicle (0.5% DMSO, twice daily for 14 days) was administered to said mice.

### Behavioral Tests

The motor function after SCI was evaluated from day 3 to day 14 after the surgery by locomotor open-field rating scale on the Basso Mouse Scale (BMS).⁶ Scoring ranged from 0 for complete paraplegia to 9 for normal function. The footprint analysis was assessed on day 14 after the surgery. The hindlimbs of the mice were dipped in red and black dye for left and right foot, respectively. The mice were then placed on a piece of drawing paper surrounded by a cage to encourage it to walk in a straight line. The stride length was defined as the distance between the middle point of two successive footprints.

### RT-qPCR Analysis

Mice were anesthetized by the inhalation of isoflurane. Spinal cords were frozen by flash freezing with liquid nitrogen and homogenized by sterile pellet pestles (Thermo Fisher Scientific, MA, USA) with 1 mL TRIzol™ Reagent (Thermo Fisher Scientific, MA, USA). Total RNA isolation, quality check, cDNA synthesis and SYBR green based quantitative real-time PCR assay were performed as described previously.⁷ The forward/reverse primer sequences for IL-1β, IL-6, TNF-α, and GAPDH were listed in Table 1. The relative expression of target genes normalized to that of glyceraldehyde 3-phosphate dehydrogenase (GAPDH) was calculated by the comparative Ct (ΔCt) and was expressed as 2−ΔCt. The relative quantity was determined by the formula: 2−ΔΔCt, in which ΔΔCt values were obtained by subtracting the ΔCt values of mice with SCI from the sham controls.

TABLE 1
Primer sequences for RT-qPCR

| | SEQ ID | |
|---|---|---|
| gene | NO | primers |
| IL-18 | 1 | Forward: |
| | | 5′-GCCCATCCTCTGTGACTCAT-3′ |
| | 2 | Reverse: |
| | | 5′-AGGCCACAGGTATTTTGTCG-3′ |
| IL-6 | 3 | Forward: |
| | | 5′-CCAGTTGCCTTCTTGGGACTG-3′ |
| | 4 | Reverse: |
| | | 5′-CAGGTCTGTTGGGAGTGGTATCC-3′ |
| TNF-& | 5 | Forward: |
| | | 5′-AAAATTCGAGTGACAAGCCTGTAG-3′ |
| | 6 | Reverse: |
| | | 5′-CCCTTGAAGAGAACCTGGGAGTAG-3′ |
| GAPDH | 7 | Forward: |
| | | 5′-TGTGTCCGTCGTGGATCTGA-3′ |
| | 8 | Reverse: |
| | | 5′-CACCACCTTCTTGATGTCATCATAC-3′ |

### Immunofluorescence Staining

After being anesthetized by Zoletil® (50 mg/kg) and Rompun® (12 mg/kg), mice were intracardially perfused with ice-cold normal saline. The spinal cord (T9-T11) was removed and soaked in ice-cold 4% paraformaldehyde. The vertebrae were then placed in cassettes for paraffin embedding. The samples were cut into sagittal section of 4 μm and placed in water bath at 42° C. The section was mounted onto slides and stored at room temperature until analysis. After deparaffinization, tissue retrieval was conducted in sodium citrate buffer (pH=6) at 90° C. and the spinal cord slices were then washed with phosphate-buffered saline (PBS). Sections were incubated in antibody dilution buffer (Roche, Basel, Swiss) at room temperature. The slides were immunostained by the following primary antibodies: chicken anti-neuronal nuclear protein (NeuN) (1:2000; Merck-Millipore, Dramstadt, Germany), rabbit anti-ionized calcium-binding adapter molecule-1 (Iba-1) (1:1000; Abcam, Cambridge, MA, USA), or mouse anti-glial fibrillary acidic protein (Gfap) (1:1000; cell signaling, Danvers, Massachusetts, USA) overnight at 4° C. Corresponding secondary antibodies conjugated with Alexa Fluor 488 or Rhodamine (Jackson Immuno Research Laboratories, West Grove, PA, USA) were applied for the visualization of immunolabelling. After being washed with PBST (Phosphate Buffered Saline with Tween® 20) and PBS, the slices were mounted on a mounting gel (DAPI Fluoromount G; Southern Biotech Birmingham, Alabama, USA). Images were acquired by a Zeiss AXIO Imager M1 microscope (Carl Zeiss, Gottingen, Germany). In the perilesional area, the fluorescence intensity of Iba-1 and Gfap were quantified by the mean optical density (mean optical density=integrated optical density (IOD)/area) by ImageJ 1.52 software (National Institutes of Health, Bethesda, Maryland, USA). The number of NeuN-positive cells in 3 sagittal sections (70 μm interval) of the spinal cord was calculated at the bilateral site of perilesional area at 20× magnification.

### Nissl Staining

After the deparaffinization, the slices were placed in PBS and then in 1% cresyl violet solution. The slices were differentiated in 95% ethyl alcohol and dehydrated in 100% alcohol. After the dehydration, slices were treated with xylene and mounted with dibutylphthalate polystyrene xylene. Regions of traumatic injury were identified by severe tissue destruction or staining loss. Six Nissl-stained sections by distance (100 μm between the sections) were selected to estimate the proportional lesion size by ImageJ 1.52 software (National Institutes of Health, Bethesda, Maryland, USA).

### Western Blot

The protein concentrations of the samples were determined by the Bio-Rad DC Protein Assay Kit (Bio-rad, Hercules, CA, USA). Protein samples (10 μg each) were diluted with loading buffer (200 mM Tris-HCl, 1.43% 2-mercaptoethanol, 0.4% bromophenol blue, and 40% glycerol) and heated at 98° C. The protein samples were then separated with 90 V for 10 minutes, followed by 130 V for 60 minutes on 12% SDS-polyacrylamide gel (for S100a10, c-caspase3 and GAPDH) or 8% SDS-polyacrylamide gel (for C3, NeuN) in running buffer (0.3% Tris base, 1.88% glycine, and 0.1% SDS). After electrophoresis, the gel was transferred onto a nitrocellulose membrane in transfer buffer (0.3% Tris base, 1.88% glycine, and 20% methanol; pH 8.3) with 300 mA for 90 minutes. Nonspecific binding to membrane was blocked by BlockPRO™ 1 Min Protein Free Blocking Buffer (Neihu, Taipei City, Taiwan) at room temperature on shaker at 25 rpm. For the detection of C3, NeuN, and GAPDH, the membrane was incubated overnight at 4° C. with antibodies for C3 (1: 200; Abcam, Cambridge, MA, USA), NeuN (1: 1000; Genetex, CA, USA), or GAPDH (1:160000; Biodesign International, Saco, Maine, USA); for the detection of S100a10 and c-caspases3, the membrane was incubated at 4° C. with S100a10 (1: 200; Abcam, Cambridge, MA, USA) or c-caspase3 (1:200; Merk-millipore, Dramstadt, Germany), all diluted in BlockPRO™ 1 Min Protein Free Blocking Buffer. The membrane was washed by TNT buffer (10 mM Tris-HCl, 150 mM NaCl, and 0.2% Tween 20; pH 7.4) and incubated with horseradish peroxidase (HRP)-conjugated anti-mouse IgG antibodies (1:5000; cell signaling, Danvers, Massachusetts, USA) or anti-rabbit IgG antibodies (1:2000; cell signaling, Danvers, Massachusetts, USA) in TNT buffer at room temperature. Bound antibodies were detected using Chemiluminescence reagent Plus (PerkinElmer Life Sciences, MA, USA) and Bio-rad ChemiDoc™ XRS+ Systems and Image Lab™ Software to obtain images under appropriate exposure time.

### Statistical Analysis

All statistical analyses were conducted by GraphPad Prism v9.1 (GraphPad Software, San Diego, CA, USA). Data of alteration of adenosine-related genes in the SCI were analyzed by the unpaired t-test. For the effects of ENT1 inhibition, except that the data of lesion volume were evaluated by the unpaired t-test, all data (i.e., BMS, stride length, RT-qPCR, immunostaining, and Western blotting) were evaluated by the one-way ANOVA. All data (i.e., BMS, stride length, lesion volume, RT-qPCR analysis, immunostaining, and Western blotting) were evaluated by the unpaired t-test. The level of significance of 0.05 was considered statistically significant.

### Results

Improvement in Functional Recovery after SCI with J4 Treatment

Please refer to FIGS. 1-7, they show J4 treatment promoted motor function recovery and reduced neuronal death in mice with SCI, wherein data are given as the mean±SEM of 3-7 animals; *p<0.05.

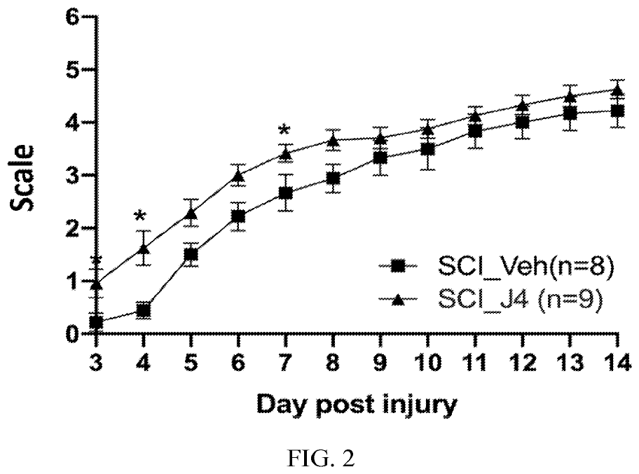

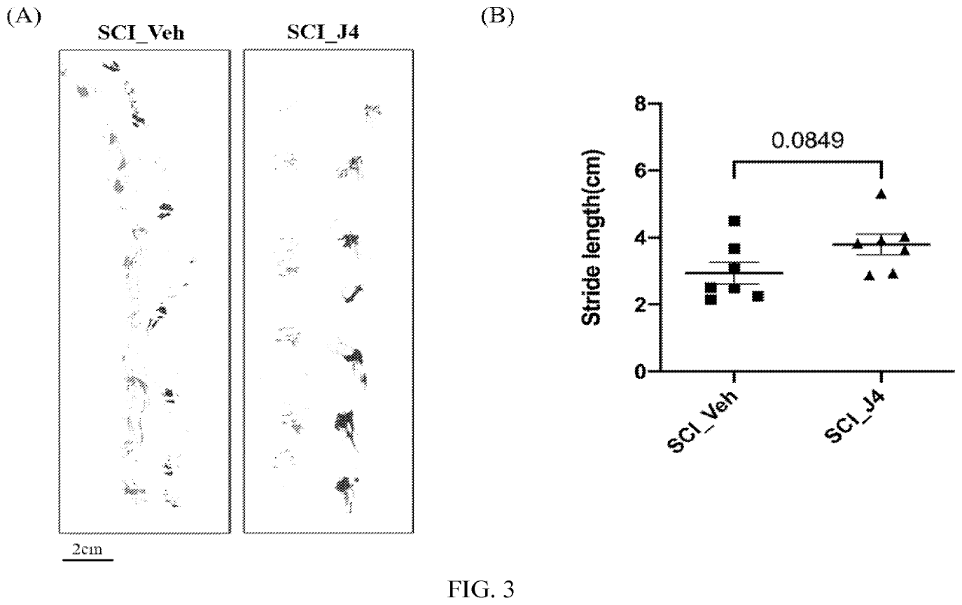

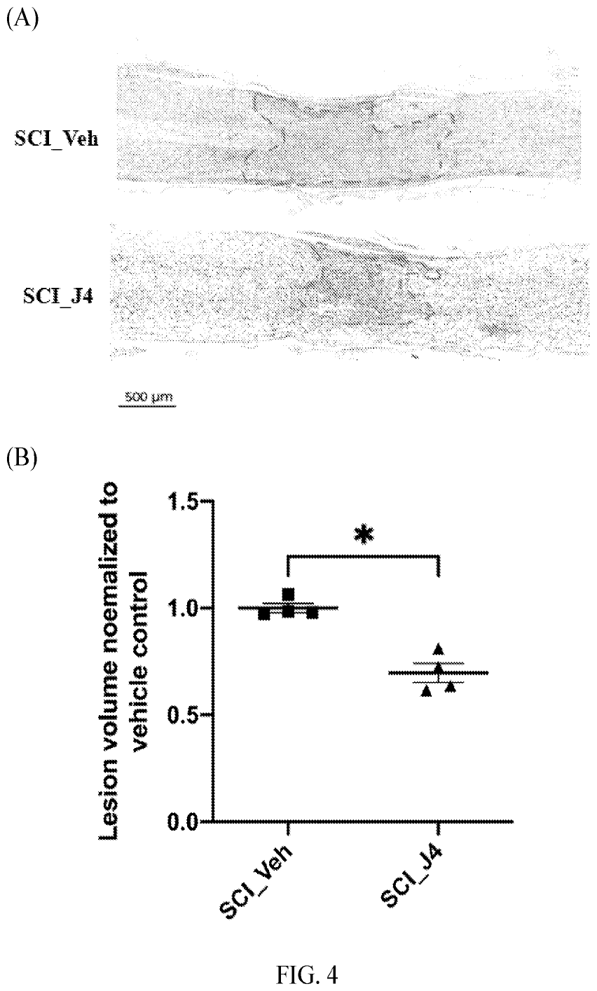

It was previously demonstrated that J4, an ENT1 inhibitor, exhibited beneficial effects on the treatment of several neurodegenerative diseases. To investigate whether J4 may also have effects on SCI, J4 was given to C57BL/6J mice with SCI, followed by the evaluation of motor function and histological changes (see FIG. 1). As shown in FIG. 2, the BMS score of mice with SCI can be improved by the treatment of J4. Further examination on the coordination of hindlimbs movements showed that the treatment of J4 improved the stride length of the mice with SCI (see FIG. 3). In terms of the lesion, the Nissl staining showed that the loss of spinal cord tissue was significantly reduced by the J4 treatment at 14 days post-injury (see FIG. 4).

These data demonstrated that the recovery of motor function and lesion in SCI can be improved by the J4 treatment.

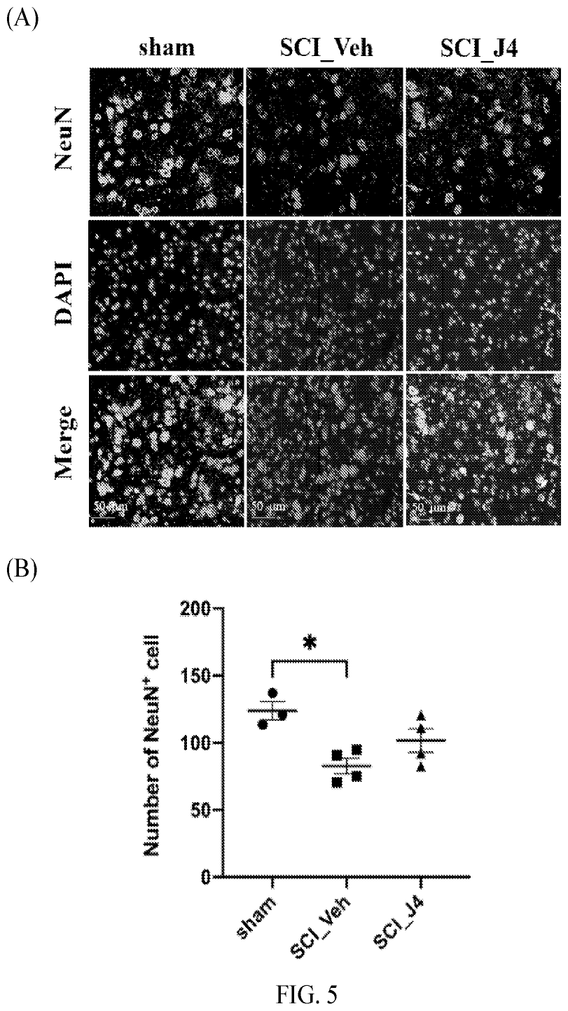

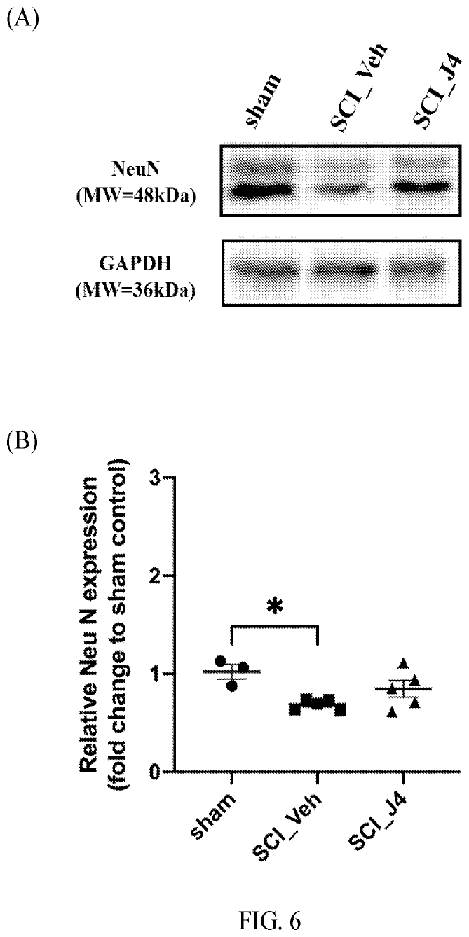

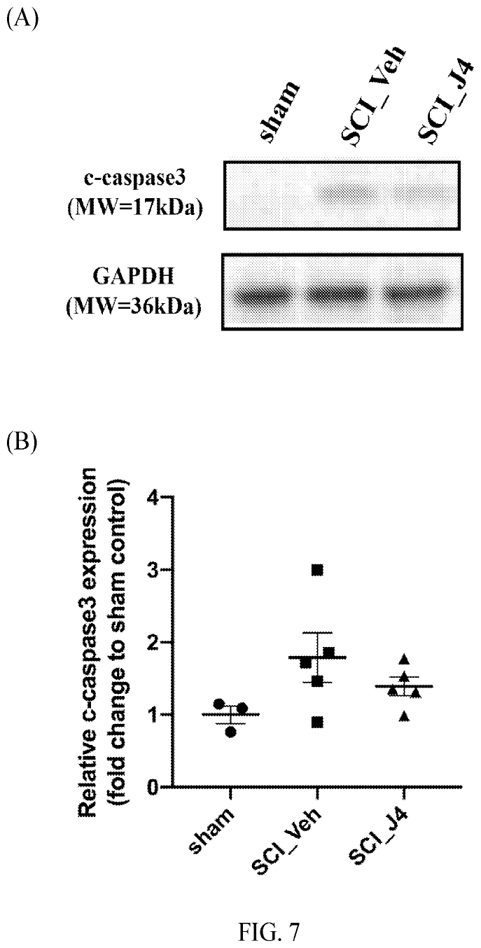

To further evaluate the preserved neuron in the perilesional area, the immunostaining and Western blot of NeuN were performed. As shown in FIG. 5, more NeuN-positive cells were identified in the perilesional area of the mice treated with J4. Likewise, protein level of NeuN was higher in the J4-treated group than in the vehicle group (see FIG. 6). Since the cleaved caspase-3(c-caspase3) is important for cell apoptosis, the protein level of c-caspase3 was examined. As shown in FIG. 7, the level of c-caspase3 was decreased by the treatment of J4 for continuous 14 days following the injury. These results showed that J4 can protect the neuronal cells from apoptosis in SCI.

Reduction of Inflammatory Responses in SCI with J4 Treatment

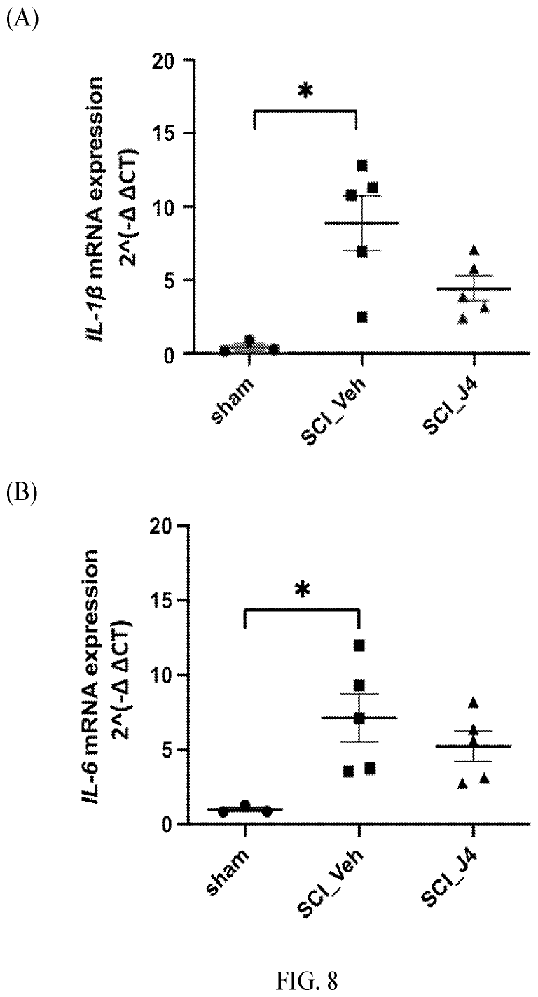

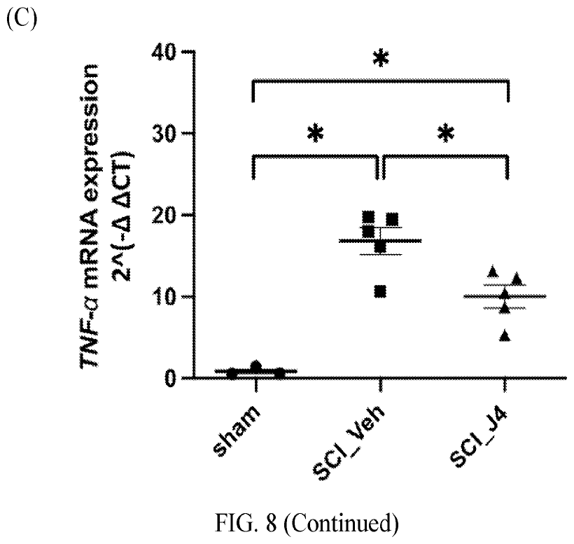

Please refer to FIGS. 8-10, they show J4 treatment reduced the inflammatory responses at perilesional area of B6 mice with SCI, wherein data are presented as mean±SEM of 3-5 animals; *p<0.05; #stands for the lesion core.

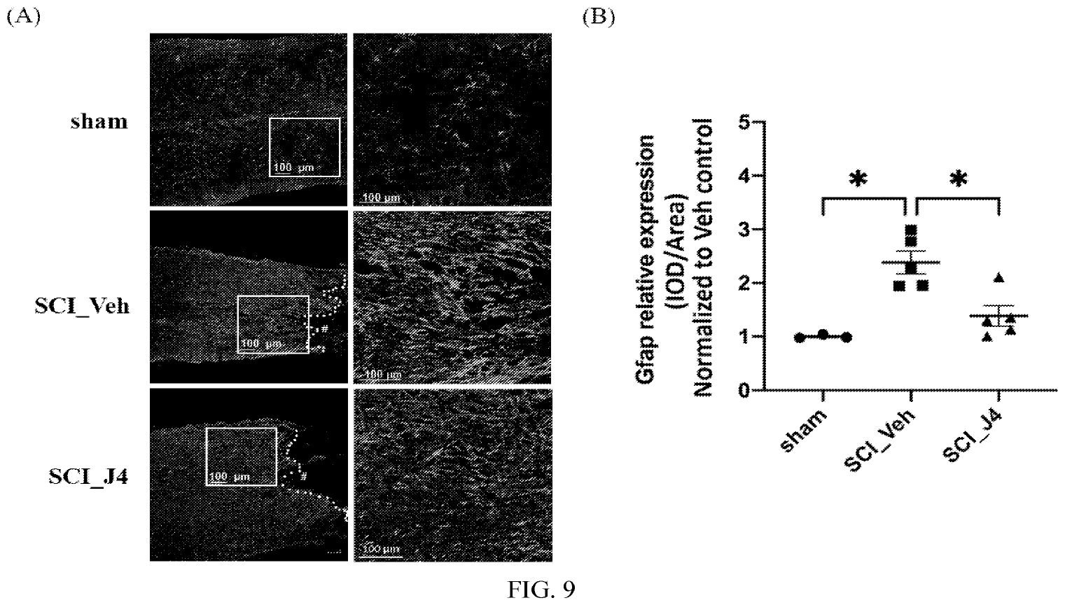

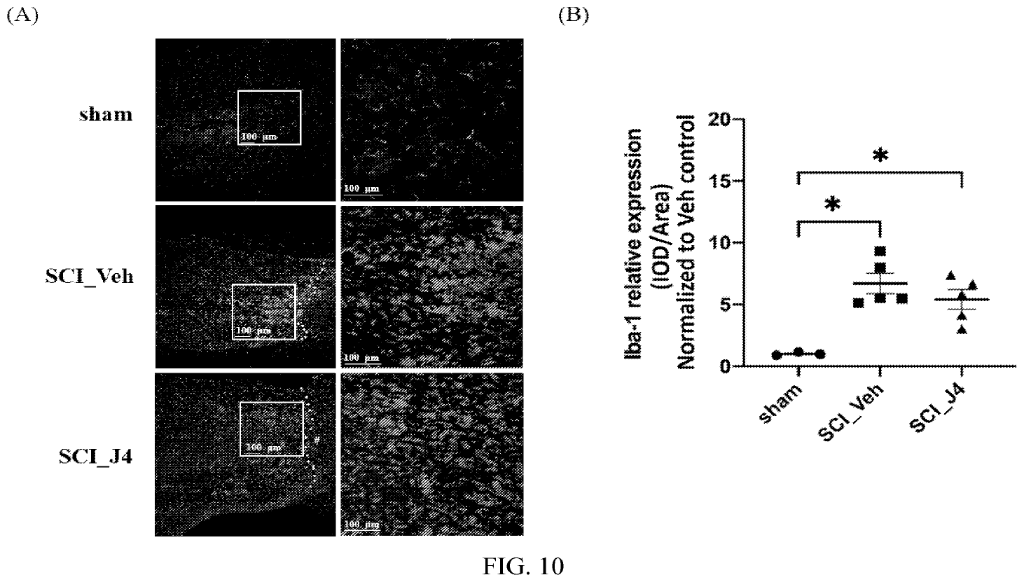

Neuroinflammation is important in the progression of SCI and IL-10, IL-6 and TNF-α are considered to be critical for post-traumatic inflammatory reaction.⁴ The effect of J4 on the expression of these cytokines at the lesion and the perilesional site of spinal cord were examined. As shown in parts (A) to (C) of FIG. 8, J4 treatment significantly reduced the expression of TNF-α, with a trend of reduction on the expression of IL-10 and IL-6, in mice with SCI. In addition to the expression of inflammatory cytokine, astrogliosis and microgliosis are important benchmarks of neuroinflammation. As shown in FIG. 9, the Gfap-positive astrocytes in the perilesional area were hypertrophied and their branches were thickened, showing morphological changes associated with the reactive status. Compared with that in the vehicle group, the average fluorescence intensity of Gfap in the J4 group was remarkably decreased at 14 days post-injury. For microgliosis, similar to astrocytes, the activated microglia undergo marked changes in cell morphology to transform from a resting state with a ramified cellular morphology to an activated state with an amoeboid-like cellular morphology. Ionized calcium-binding adaptor protein-1 (Iba-1), a 17-kDa actin-binding protein, is widely employed as an immunohistochemical marker for both microglia and macrophage. As shown in FIG. 10, the intensity of Iba-1-positive cells was lower in the J4-treated group, compared with the vehicle-treated group. These findings showed that J4 can reduce the inflammatory responses, including the expression of inflammatory cytokines and the activation of astrocytes and microglia/macrophage at the perilesional site of the spinal cord.

Modulation for Phenotypes of Astrocytes in SCI with J4 Treatment

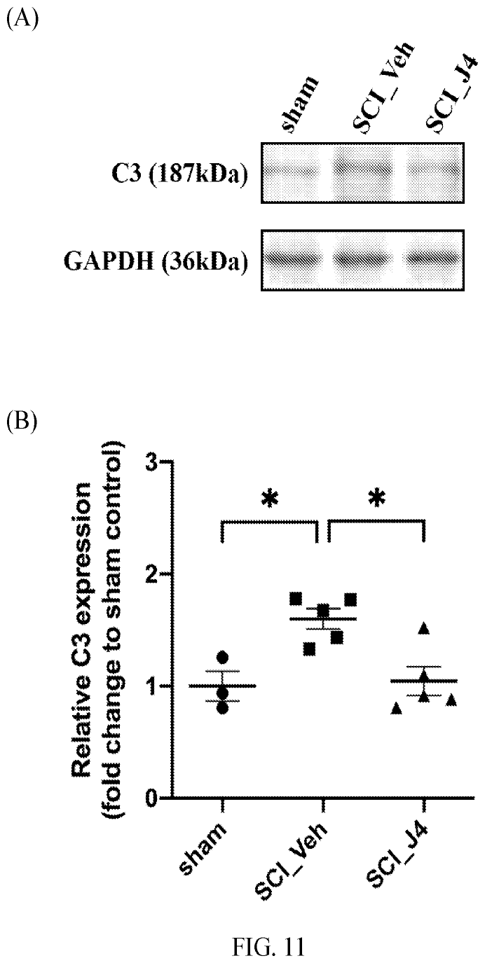

Please refer to FIGS. 11-13, they show J4 treatment modulated the phenotypes of astrocytes and microglia/macrophage at T9-T11 of B6 mice with SCI, wherein data are presented as mean±SEM of 3-5 animals; *p<0.05.

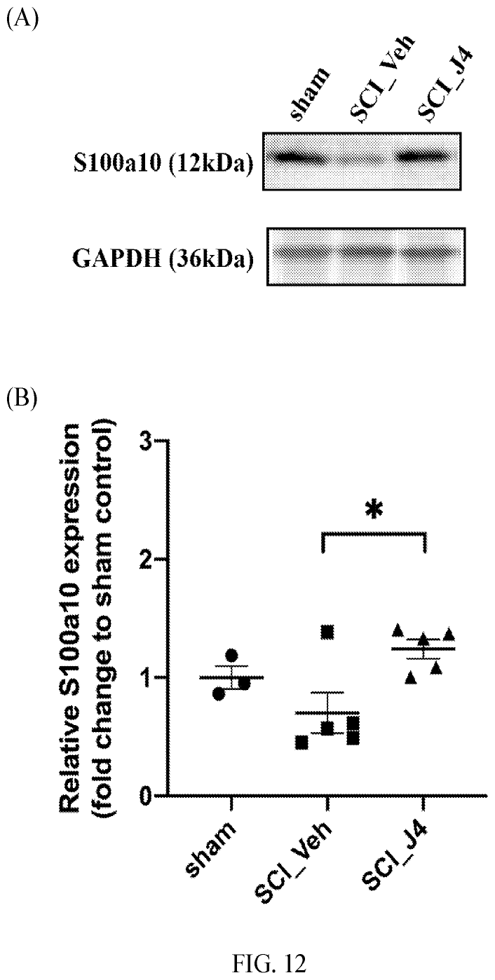

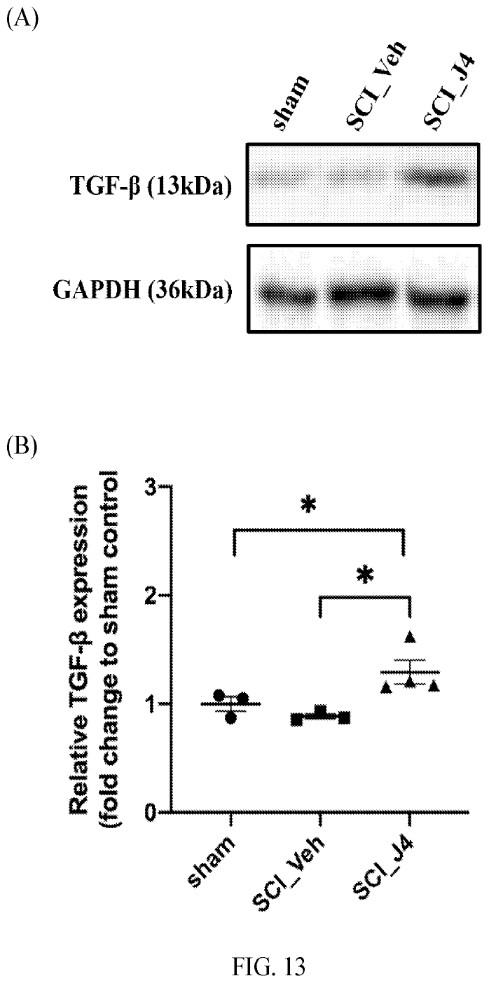

The activated astrocytes can develop to different phenotypes, in which the A1 astrocyte is considered to be neurotoxic and A2 astrocyte is restorative. Accordingly, the expression of C3 and S100a10, the makers of A1 and A2 astrocytes, respectively⁸, were examined. As a result, protein levels of C3 and S100a10 were significantly decreased and increased, respectively, by the treatment of J4 (see FIGS. 11 and 12). These findings showed that J4 treatment can elevate A2 astrocytes expression and lower the expression of A1 astrocytes. As astrocytes can produce transforming growth factor β (TGF-β) that has been implicated in inducing axon formation and neurogenesis 9, the level of TGF-β was examined. As shown in FIG. 13, TGF-β protein was significantly increased by J4 treatment.

Treatment with JMF 1907 for SCI

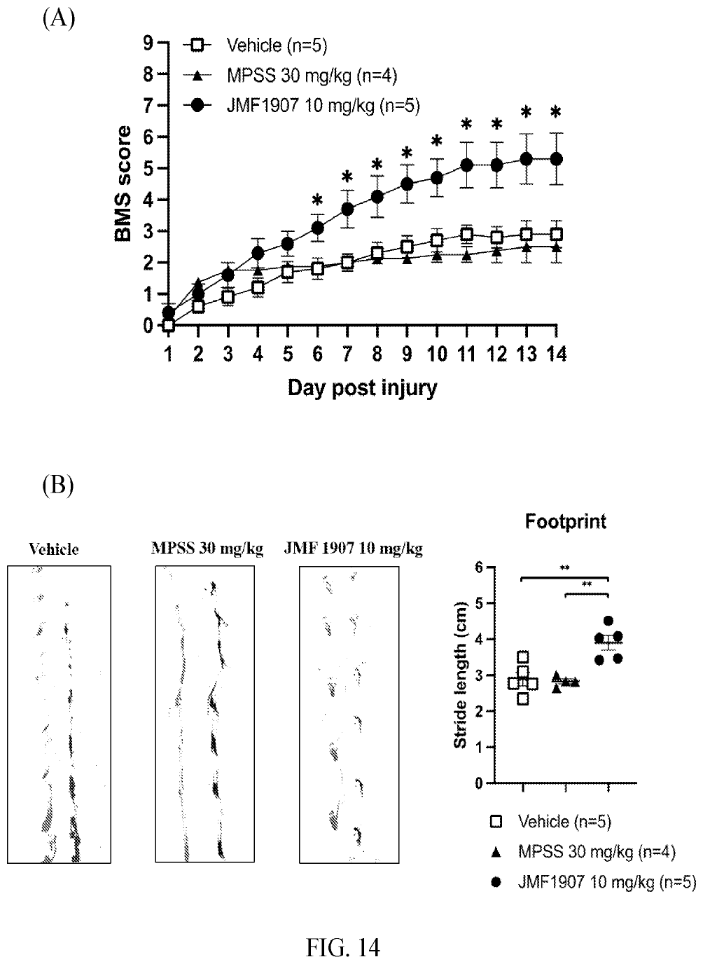

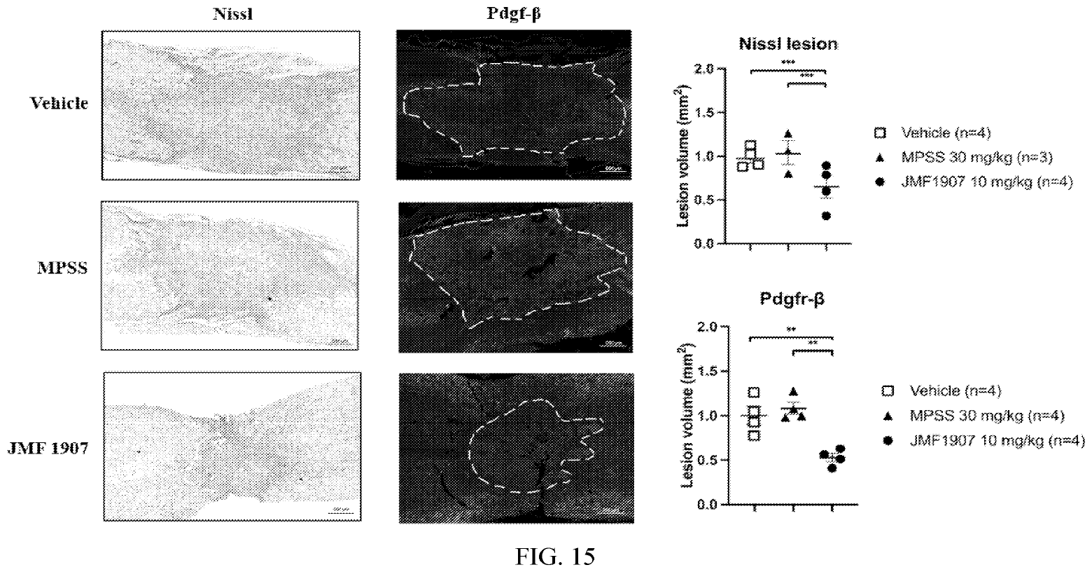

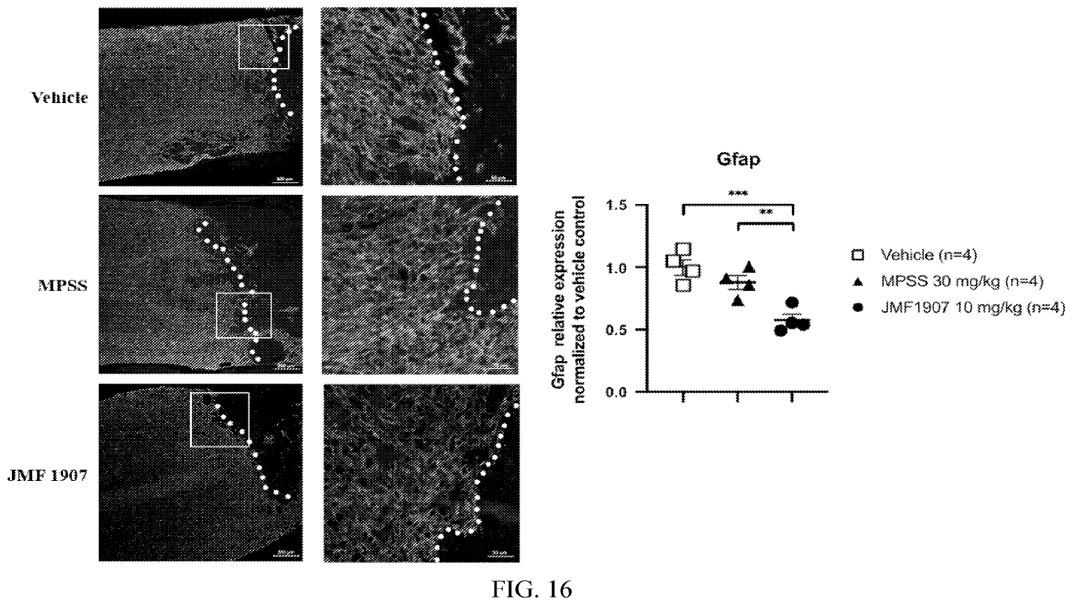

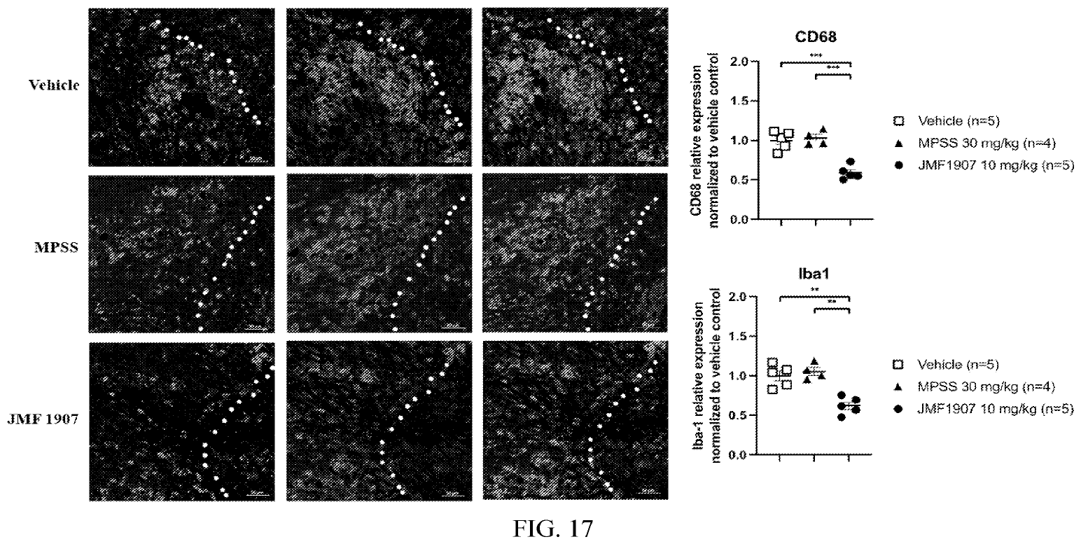

Similarly, the results showed that the treatment with JMF1907 can significantly improve the motor function (in terms of the BMS score) (part (A) of FIG. 14) and the stride length (part (B) of FIG. 14) of the mice with SCI. Yet, treatments with MPSS did not improve the motor function of mice with SCI. The immunofluorescence analysis and Nissl staining showed that JMF1907 treatment can significantly reduce the lesion of the SCI (FIG. 15), consistent with the findings of motor function measurement. In addition, JMF1907 treatments significantly reduced the levels of Gfap (a marker for reactive astrocytes) (FIG. 16) and Iba-1 ad CD68 (both are markers for microglia) (FIG. 17) in mice with SCI, showing the beneficial effects of JMF1907 on reducing neuroinflammation in SCI.

### Discussion

Following the primary damage on the spinal cord, inflammation plays an important role towards the development of secondary injury that may lead to neuronal death.⁴ While methylprednisolone has been applied to reduce the inflammatory responses in SCI, its use is highly controversial and is not approved by the US Food and Drug Administration (FDA) for this indication.¹⁰ To date, the treatment of SCI is still considered to be an unmet medical need. Upon the increase of extracellular adenosine level, ENTs plays an important role in mediating cellular uptake of adenosine. The present invention showed that J4 used as pharmacological inhibition of ENT1 can attenuate the inflammation, reduce the lesion, and improve functional recovery in mice of thoracic spinal cord contusion model. These findings suggest that J4 is a potential candidate for treating SCI.

ENTs play important roles in controlling extracellular level of adenosine. At the spinal cord, selective inhibition of ENT1 by NBMPR can modulate glutamatergic synaptic transmission via AiR activation.¹¹ The present invention further demonstrated that the inhibition of ENT1 exhibited anti-inflammation effect and improve functional recovery in SCI.

Neuroinflammation is important in the progression of SCI, in which targeting inflammation may provide a way to improve neuronal function and the outcomes of SCI.¹² In SCI, neuroinflammation involves microgliosis, the infiltration of macrophages, and astrogliosis in SCI. Both the expression of Iba-1 (for microglia/macrophage) and Gfap (for astrocytes) were increased in SCI. The activation of microglia/macrophage and astrocytes can be reduced by pharmacological inhibition. Similar finding in astrocyte has been reported, in which Gfap expression is reduced in ENT1 null mice.¹³

In terms of the phenotype of the activated astrocytes, the present results showed that J4 treatment significantly reduced protein level of C3 (A1 marker), compared with untreated SCI mice. On the other hand, J4 significantly increased protein level of S100a10. A1 astrocytes have shown neurotoxic effect to oligodendrocytes and neurons in vitro, whereas A2 astrocytes promote neuronal survival and tissue repair.¹⁴⁻¹⁵ It was previously reported that the functional recovery in mice with SCI may be improved by reducing astrocytes with A1 phenotype¹⁶; the increase of A2 astrocytes may provide beneficial effect on reducing the pro-inflammatory cytokines production¹⁷. Given that increasing A2 astrocytes or decreasing A1astrocytes may be beneficial to the injury¹⁸, the modulation of A1/A2 phenotype by J4 treatment support the beneficial effect of J4 in the treatment of SCI.

### Conclusion

According to the results above, they showed that mice administrated with the compounds of the present invention had higher BMS score and longer stride length, compared with the controls. The treatment with the compounds of the present invention significantly reduced the mRNA levels of IL-1β, IL-6 and TNF-α and the activation of astrocytes and microglia/macrophage at the perilesional site of the spinal cord on 14 DPI. Along with reduced size of lesion volume, more preserved neurons were identified in the perilesional area of J4-treated mice treated with the compounds of the present invention.

Taken together, the treatment with the compounds of the present invention can ameliorate neuroinflammation, reduce the lesion, and improve motor function recovery in mice with thoracic spinal cord injury. In addition, the compounds of the present invention can modulate the A1/A2 phenotypes of the activated astrocytes, that may facilitate neural regeneration. These results suggest that the compounds of the present application are potential candidates for the treatment of SCI.

### REFERENCES

1. James S L, Theadom A, Ellenbogen R G, Bannick M S, Montjoy-Venning W, Lucchesi L R, et al. Global, regional, and national burden of traumatic brain injury and spinal cord injury, 1990-2016: a systematic analysis for the Global Burden of Disease Study 2016. Lancet Neurol. 2019; 18:56-87.
2. Beattie, M. S., Hermann, G. E., Rogers, R. C. and Bresnahan, J. C. (2002). Cell death in models of spinal cord injury. Prog Brain Res 137, 37-47.
3. David S, Kroner A. Inflammation and secondary damage after spinal cord injury. In: So KF, Xu XM, editors. Neural Regeneration. Academic Press; 2015. p. 245-261.
4. Hausmann ON. Post-traumatic inflammation following spinal cord injury. Spinal Cord. 2003; 41:369-378.
5. Okada S, Nakamura M, Katoh H, Miyao T, Shimazaki T, Ishii K, et al. Conditional ablation of Stat3 or Socs3 discloses a dual role for reactive astrocytes after spinal cord injury. Nat Med. 2006; 12:829-834.
6. Basso D M, Fisher L C, Anderson A J, Jakeman L B, McTigue D M, Popovich P G. Basso Mouse Scale for locomotion detects differences in recovery after spinal cord injury in five common mouse strains. J Neurotrauma. 2006; 23:635-659.
7. Lai P H, Wang T H, Zhang N Y, Wu K C, Yao C J, Lin C J. Changes of blood-brain-barrier function and transfer of amyloid beta in rats with collagen-induced arthritis. J Neuroinflamation. 2021; 18:35.
8. Boghdadi A G, Teo L, Bourne J A. The neuroprotective role of reactive astrocytes after central nervous system injury. J Neurotrauma. 2020; 37:681-691.
9. Villapol S, Logan T T, Symes A J. Role of TGF-β signaling in neurogenic regions after brain injury. In: Wislet-Gendebien, S, editor. Trends in cell signaling pathways in neuronal fate decision. IntechOpen; 2013. p.1-36.
10. Hurlbert R J, Hadley M N, Walters B C, Aarabi B, Dhall S S, Gelb D E, et al. Pharmacological therapy for acute spinal cord injury. Neurosurgery. 2013; 72 Suppl 2:93-105.
11. Ackley, M. A., Governo, R. J., Cass, C. E., Young, J. D., Baldwin, S. A. and King, A. E. (2003). Control of glutamatergic neurotransmission in the rat spinal dorsal horn by the nucleoside transporter ENT1. J Physiol 548, 507-517.
12. Orr M B, Gensel J C. Spinal cord injury scarring and inflammation: therapies targeting glial and inflammatory responses. Neurotherapeutics. 2018; 15:541-553.
13. Hinton D J, Lee M R, Jang J S, Choi D S. Type 1 equilibrative nucleoside transporter regulates astrocyte-specific glial fibrillary acidic protein expression in the striatum. Brain Behav. 2014; 4:903-914.
14. Qian, D., Li, L., Rong, Y., Liu, W., Wang, Q., Zhou, Z., Gu, C., Huang, Y., Zhao, X., Chen, J., Fan, J. and Yin, G. (2019). Blocking Notch signal pathway suppresses the activation of neurotoxic A1 astrocytes after spinal cord injury. Cell Cycle 18, 3010-3029.
15. Fujita, A., Yamaguchi, H., Yamasaki, R., Cui, Y., Matsuoka, Y., Yamada, K. I. and Kira, J. I. (2018). Connexin 30 deficiency attenuates A2 astrocyte responses and induces severe neurodegeneration in a 1-methyl-4-phenyl-1,2,3,6-tetrahydropyridine hydrochloride Parkinson's disease animal model. J Neuroinflammation 15, 227.
16. Wang L, Pei S, Han L, Guo B, Li Y, Duan R, et al. Mesenchymal stem cell-derived exosomes reduce A1 astrocytes via downregulation of phosphorylated NFκB P65 subunit in spinal cord injury. Cell Physiol Biochem. 2018; 50:1535-1559.
17. Neal M, Luo J, Harischandra D S, Gordon R, Sarkar S, Jin H, et al. Prokineticin-2 promotes chemotaxis and alternative A2 reactivity of astrocytes. Glia. 2018; 66:2137-2157.
18. Chan, S. J., Niu, W., Hayakawa, K., Hamanaka, G., Wang, X., Cheah, P. S., Guo, S., Yu, Z., Arai, K., Selim, M. H., Kurisawa, M., Spector, M. and Lo, E. H. (2019). Promoting Neuro-Supportive Properties of Astrocytes with Epidermal Growth Factor Hydrogels. Stem Cells Transl Med 8, 1242-1248.

What is claimed is:

1. A method of treating spinal cord injury, comprising administering to a subject in need thereof a compound of formula (I), (II) or (III): 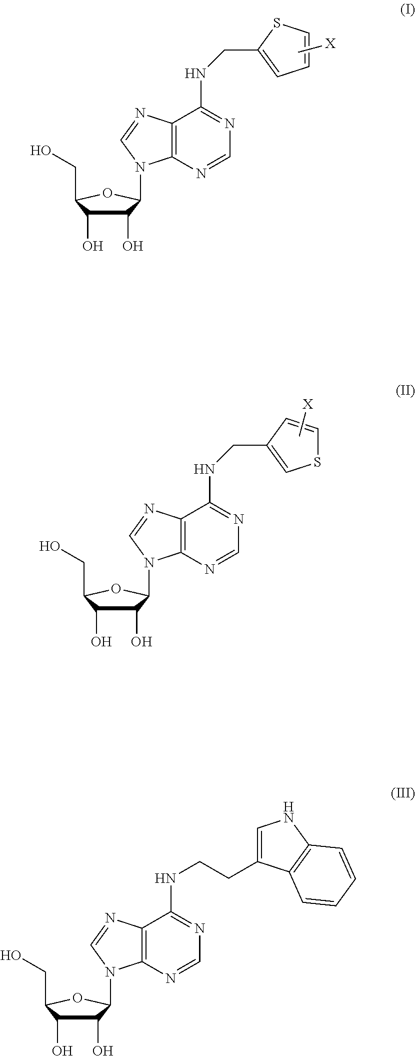
a pharmaceutically acceptable salt thereof, or a composition thereof, wherein X is halogen.

2. The method of claim 1, wherein the compound is selected from the group consisting of N⁶-[(3-halothien-2-yl)methyl]adenosine, N⁶-[(4-halothien-2-yl)methyl]adenosine, and N⁶-[(5-halothien-2-yl)methyl]adenosine.

3. The method of claim 2, wherein the compound is selected from the group consisting of N⁶-[(5-iodothien-2-yl)methyl]adenosine, N⁶-[(4-iodothien-2-yl)methyl]adenosine, N⁶-[(3-iodothien-2-yl)methyl]adenosine, N⁶-[(5-bromothien-2-yl)methyl]adenosine, N⁶-[(4-bromothien-2-yl)methyl]adenosine, N⁶-[(3-bromothien-2-yl)methyl]adenosine, N⁶-[(5-chlorothien-2-yl)methyl]adenosine, N⁶-[(4-chlorothien-2-yl)methyl]adenosine, and N⁶-[(3-chlorothien-2-yl)methyl]adenosine.

4. The method of claim 1, wherein the compound is selected from the group consisting of N⁶-[(2-halothien-3-yl)methyl]adenosine, N⁶-[(4-halothien-3-yl)methyl]adenosine, and N⁶-[(5-halothien-3-yl)methyl]adenosine.

5. The method of claim 4, wherein the compound is selected from the group consisting of N⁶-[(2-iodothien-3-yl)methyl]adenosine, N⁶-[(4-iodothien-3-yl)methyl]adenosine, N⁶-[(5-iodothien-3-yl)methyl]adenosine, N⁶-[(2-bromothien-3-yl)methyl]adenosine, N⁶-[(4-bromothien-3-yl)methyl]adenosine, N⁶-[(5-bromothien-3-yl)methyl]adenosine, N⁶-[(2-chlorothien-3-yl)methyl]adenosine, N⁶-[(4-chlorothien-3-yl)methyl]adenosine, and N⁶-[(5-chlorothien-3-yl)methyl]adenosine.

6. The method of claim 1, wherein the compound, a pharmaceutically acceptable salt thereof, or a composition thereof is administered by an oral, intravenous, intramuscular, subcutaneous, intraperitoneal or topical route.

7. The method of claim 1, wherein the composition further comprises a pharmaceutically acceptable carrier, excipient or vehicle.

8. The method of claim 1, wherein the treating spinal cord injury comprises improving functional recovery after spinal cord injury.

9. The method of claim 1, wherein the treating spinal cord injury comprises reducing neuroinflammation in neuro after spinal cord injury.

10. The method of claim 1, wherein the treating spinal cord injury comprises modulating the phenotypes of astrocytes and microglia/macrophage in spinal cord injury.

11. A composition for use in a method of treating spinal cord injury, comprising administering to a subject in need thereof the composition comprising a compound of formula (I), (II) or (III): 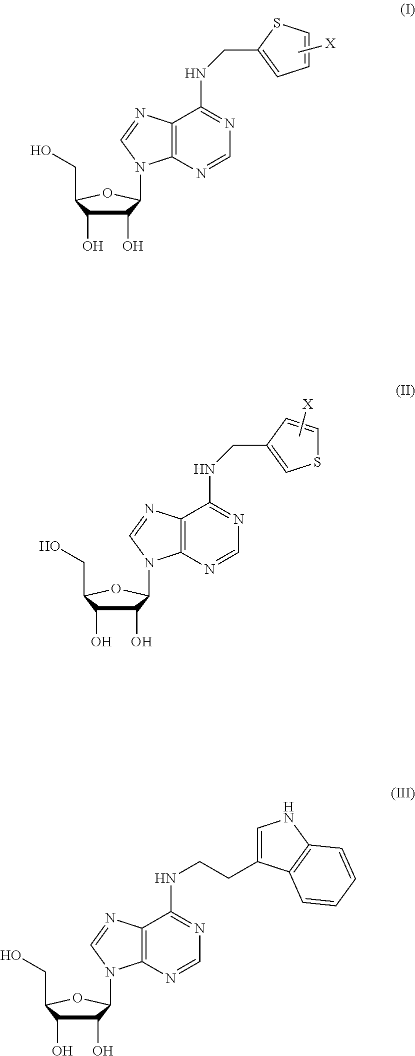
or a pharmaceutically acceptable salt thereof, wherein X is halogen.

12. The composition for use of claim 11, wherein the compound is selected from the group consisting of N⁶-[(3-halothien-2-yl)methyl]adenosine, N⁶-[(4-halothien-2-yl)methyl]adenosine, and N⁶-[(5-halothien-2-yl)methyl]adenosine.

13. The composition for use of claim 12, wherein the compound is selected from the group consisting of N⁶-[(5-iodothien-2-yl)methyl]adenosine, N⁶-[(4-iodothien-2-yl)methyl]adenosine, N⁶-[(3-iodothien-2-yl)methyl]adenosine, N⁶-[(5-bromothien-2-yl)methyl]adenosine, N⁶-[(4-bromothien-2-yl)methyl]adenosine, N⁶-[(3-bromothien-2-yl)methyl]adenosine, N⁶-[(5-chlorothien-2-yl)methyl]adenosine, N⁶-[(4-chlorothien-2-yl)methyl]adenosine, and N⁶-[(3-chlorothien-2-yl)methyl]adenosine.

14. The composition for use of claim 11, wherein the compound is selected from the group consisting of N⁶-[(2-halothien-3-yl)methyl]adenosine, N⁶-[(4-halothien-3-yl)methyl]adenosine, and N⁶-[(5-halothien-3-yl)methyl]adenosine.

15. The composition for use of claim 14, wherein the compound is selected from the group consisting of N⁶-[(2-iodothien-3-yl)methyl]adenosine, N⁶-[(4-iodothien-3-yl)methyl]adenosine, N⁶-[(5-iodothien-3-yl)methyl]adenosine, N⁶-[(2-bromothien-3-yl)methyl]adenosine, N⁶-[(4-bromothien-3-yl)methyl]adenosine, N⁶-[(5-bromothien-3-yl)methyl]adenosine N⁶-[(2-chlorothien-3-yl)methyl]adenosine, N⁶-[(4-chlorothien-3-yl)methyl]adenosine, and N⁶-[(5-chlorothien-3-yl)methyl]adenosine.

16. The composition for use of claim 11, wherein the composition is administered by an oral, intravenous, intramuscular, subcutaneous, intraperitoneal, or topical route.

17. The composition for use of claim 11, wherein the composition further comprises a pharmaceutically acceptable carrier, excipient or vehicle.

18. The composition for use of claim 11, wherein the treating spinal cord injury comprises an improving functional recovery after spinal cord injury.

19. The composition for use of claim 11, wherein the treating spinal cord injury comprises reducing neuroinflammation after spinal cord injury.

20. The composition for use of claim 11, wherein the treating spinal cord injury comprises modulating the phenotypes of astrocytes and microglia/macrophage in spinal cord injury.
